(19) United States
(12) Patent Application Publication
(10) Pub. No.: US 2026/0215774 A1
(43) Pub. Date: Jul. 30, 2026

(54) SUTURE APPLICATOR DEVICE AND SYSTEM

(71) Applicant: Brian Hormozi, Lancaster, CA (US)

(72) Inventor: Brian Hormozi, Lancaster, CA (US)

(21) Appl. No.: 19/039,703

(22) Filed: Jan. 28, 2025

(51) Int. Cl. A61B 17/04; A61B 17/00; A61B 17/06

(52) U.S. Cl. CPC A61B 17/0491; A61B 17/06114; A61B 2017/00367; A61B 2017/00424; A61B 2017/00862; A61B 2017/061

(57) ABSTRACT

A device and system for suturing lacerations in human and animal tissue is provided. The device comprises a shell defining an interior and an exterior, configured to securely mount a hollow surgical needle. A mechanical actuator is disposed on the exterior of the shell and operatively connected to a stitch driver housed within the interior. The device further includes a mechanism for feeding a stitch clip containing a plurality of elastic, biocompatible filaments arranged as individual stitches or sutures and removably attached to a spine into the interior of the shell. The mechanical actuator is operable to advance the stitch driver through the hollow surgical needle, engaging one or more of the filaments and deploying them through the needle to secure opposing sides of lacerated tissue.

### GOVERNMENT CONTRACT

Not applicable.

### CROSS-REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to medical instruments for treating lacerations in human and animal tissues, and more particularly, to a mechanical stitch applicator and system.

### BACKGROUND

Conventional methods for suturing lacerations in human and animal tissue typically involves either hand suturing with absorbable or non-absorbable sutures or the use of a staple gun. Hand suturing is widely regarded as the preferred method for achieving precise wound closure and minimizing scarring. However, it is time-intensive and requires significant skill and effort, which can be a drawback in high-pressure or resource-limited settings.

Efforts to improve laceration repair have led to the development of various methods and devices, each addressing specific challenges but often falling short in critical areas. Stapling devices, such as U.S. Pat. No. 5,865,361 to Milliman et al. and European Pat. No. 2,529,675 to Smith et al. for example, have been introduced to expedite wound closure, particularly for large or contaminated lacerations. While efficient, staples often cause significant scarring, lack precision for precise alignment of wound borders, and are unsuitable for delicate or aesthetically sensitive areas.

Adhesive skin closures, such as medical-grade glues and adhesive strips, provide a non-invasive option for small, superficial wounds. Some of these are addressed, for example and not limitation in U.S. Pat. No. 3,269,903 to von Fieandt et al and U.S. Pat. No. 3,987,000 to Gleichenhagen et al. However, these dressings are best suited for treating relatively minor, low-tension injuries and can fail under moisture or bodily fluids, making them unsuitable for more complex or deeper lacerations.

Barbed sutures, such as that described in U.S. Pat. No. 11,723,654 to Ruff et al. have been designed to eliminate knot tying and reduce suturing time and offer self-anchoring capabilities. However, the barbs can tear delicate, already damaged tissue under excessive tension and require careful handling, which may still be time-consuming.

Thus, although various proposals have been made to solve problems associated with laceration repair, none of those in existence combine the characteristics of the present invention. Therefore, there remains a need for a device and system that efficiently, precisely, adaptably, and aesthetically suture tissue lacerations for a wide range of medical and veterinary applications.

### SUMMARY

The present disclosure is directed to a device and system for suturing open lacerations in animal and human tissue with individual, ready-made biocompatible stitches. A suture applicator may be configured in any manner to accommodate both left-handed and right-handed users may be further adapted to facilitate the advancement of individual stitches via a curved, hollow surgical needle across the borders of a laceration from underneath a subject's dermis. It is contemplated that the device and system will avoid or eliminate reliance on auxiliary tools, such as a needle holder and fine suturing scissors, typically required to tension and tie suture threads.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In accordance with one embodiment, a suture applicator system may comprise a suture applicator, stitch clip comprising a plurality of elastic, biocompatible filaments as individual stitches feedable into the suture applicator, and a hollow surgical needle, such as a biocompatible curved needle configured to receive individual stitches therethrough.

Note that “stitch” and “suture” may be used interchangeably throughout this disclosure to mean thread or row of threads used to close a laceration.

The shell may define an inside and an outside configured to securely mount the hollow surgical needle. Means for driving individual stitches through the hollow surgical needle are provided. For instance, in some embodiments, a mechanical actuator may be disposed on the outside of the shell and operatively connected to a stitch driver housed within the interior of the shell. Likewise, means for feeding the stitch clip into the interior of the shell may be provided. In some embodiments, such means comprises a groove disposed on a portion of the applicator which is configured to guide the stitch clip inside the applicator to align a stitch with an open end of the hollow surgical needle. That is, in some embodiments, a mechanical actuator is operative to advance the stitch driver into an end of the hollow surgical needle, such that the stitch driver engages one or more of the elastic, biocompatible filaments from the stitch clip and deploys the filament through the hollow surgical needle into opposing sides of lacerated human or animal tissue.

The hollow surgical needle may be any shape or size as desired to needed to guide a stitch from an outside of one laceration border, underneath the dermis, and then to an outside of an opposing laceration border. In some embodiments, a front end of the hollow surgical needle may be beveled, or sharpened to an angular tip to reduce force needed to puncture tissue and further reduce the amount that the needle may deflect when inserted into tissue. The particular bevel length, bevel angle and inclination angle will not limit the invention.

Once a user, such as veterinarian or physician, guides the hollow surgical needle from outside of one laceration border, underneath the dermis, and then to an outside of an opposing laceration border, such user may engage the means for driving individual stitches in order to push an individual suture from a stitch clip engaged with the stitch applicator through one end of the hollow surgical needle and out the opposing beveled end.

Each filament comprising the individual stitches themselves comprise an elastic, biocompatible, and medically acceptable structure with a central connecting segment and two opposing elongate legs extending from the central segment. When deployed, the first elongate leg is driven through the hollow surgical needle and emerges on the exterior of one side of the lacerated tissue. The central segment is positioned beneath the tissue, preferable underneath the dermis, while the second elongate leg emerges on the exterior of the opposing side of the laceration. This configuration secures the tissue by anchoring the filament on both external sides of the laceration and maintaining the central segment beneath the tissue.

The filament may comprise any suitable biocompatible material as needed or desired to repair lacerations in human or animal tissues. For example, absorbable monofilament sutures may be utilized for being characterized by high flexibility and elasticity. These features may be useful when suturing particularly fragile tissues. Braided or twisted multifilament sutures are also contemplated and may be selected for their higher memory and low bacterial adhesion. As above, the particular type, size, construction, and quality of the filament selected to define individual stitches will not limit the invention and may vary depending on the needs and preferences of the user and the lacerated subject.

In some embodiments, the mechanical actuator of the device may be integrated with a depressable button or trigger accessible from the outside of the applicator shell. It is contemplated that when the button or trigger is engaged, it may directly activate any internal mechanism to advance the stitch driver, deploy an individual filament from the clip through the hollow surgical needle, and reset the system for subsequent operations. This integration simplifies the operation of the device, enabling precise control over the deployment process while reducing the number of components required. The compact configuration enhances ergonomic handling, making the device intuitive and efficient for suturing applications in both human and animal tissue.

In some embodiments, the suture applicator may be a component in a kit or system in which one or more stitch clips and/or surgical needles are provided in sterile packaging.

In some embodiments, the suture applicator may itself be sterilizable for reuse. In some embodiments, plastic and other single use or sterilizable barriers may be provided where needed to prevent contamination of the suture applicator.

It is contemplated that providing a suture applicator device and system according to the disclosure and claims provided below may improve the efficiency and precision of suturing lacerations, reduce procedure time compared to traditional hand suturing methods, and offer a less invasive alternative to staples that minimizes scarring and facilitates more effective wound healing.

The device and system may further enhance the aesthetic and functional outcomes of laceration repair while healing is in process as well by enabling sutures to be placed within the dermis, leaving no visible external sutures.

Additionally, the system may accommodate both simple and complex lacerations, providing versatility for use in various medical and veterinary settings.

One or more of the above-disclosed embodiments, in addition to certain alternatives, are provided in further detail below with reference to the attached figures. The disclosed subject matter is not, however, limited to any particular embodiment disclosed.

### BRIEF DESCRIPTION OF THE DRAWINGS

FIG. 1 shows a perspective view of one embodiment of the suture applicator device and system.

FIG. 2 shows an exemplary exploded view of one embodiment of the suture applicator device and system.

FIG. 3 shows a close-up view of an exemplary filament feeder according to one exemplary embodiment of the suture applicator device and system.

FIG. 4 shows one exemplary embodiment of an exemplary suture applicator device and system in use.

FIG. 5A shows another exemplary embodiment of an exemplary suture applicator device and system in use.

FIG. 5B shows exemplary sutures applied with an exemplary suture applicator device and system in accordance with one embodiment.

FIG. 6 shows a perspective view of an alternative embodiment of the suture applicator device and system.

The disclosed embodiments may be better understood by referring to the figures in the attached drawings, as provided below. The attached figures are provided as non-limiting examples for providing an enabling description of the method and system claimed. Attention is called to the fact, however, that the appended drawings illustrate only typical embodiments of this invention and are therefore not to be considered as limiting of its scope. One skilled in the art will understand that the invention may be practiced without some of the details included in order to provide a thorough enabling description of such embodiments. Well-known structures and functions have not been shown or described in detail to avoid unnecessarily obscuring the description of the embodiments.

### DETAILED DESCRIPTION

Having summarized various aspects of the present disclosure, reference will now be made in detail to that which is illustrated in the drawings. While the disclosure will be described in connection with these drawings, there is no intent to limit it to the embodiment or embodiments disclosed herein. Rather, the intent is to cover all alternatives, modifications and equivalents included within the spirit and scope of the disclosure as defined by the appended claims.

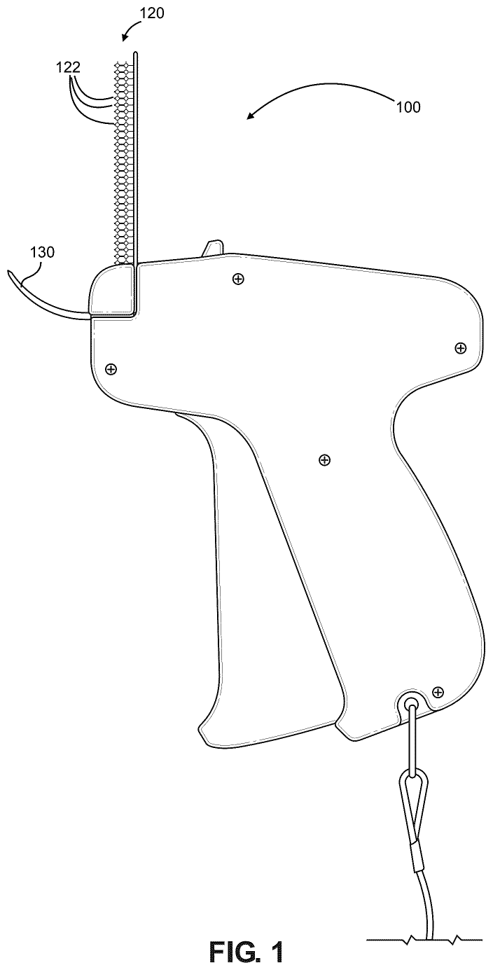

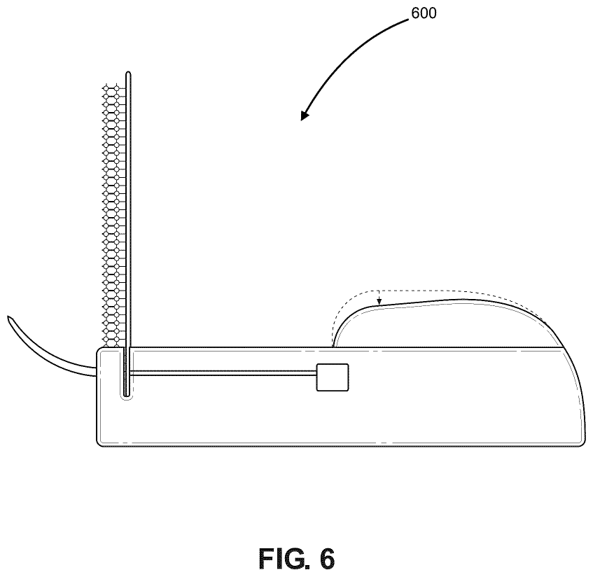

With reference to FIG. 1, a suture applicator system 100 may comprise a suture applicator 110, stitch clip 120 comprising a plurality of elastic, biocompatible filaments 122 as individual stitches feedable into the suture applicator 110, and a hollow surgical needle 130, such as a biocompatible curved needle configured to receive individual stitches therethrough. FIG. 6 shows an alternative exemplary embodiment of a suture applicator system 600 contemplated by the disclosure. Of course, the suture applicator device and system may take numerous forms as desired and needed for ergonomics and ease of use by any individual applying stitches or sutures to repair a laceration in human or animal tissue. Thus, the particular appearance or shape of any components of the suture applicator device and system are for illustrative purposes only and shall not limit the invention.

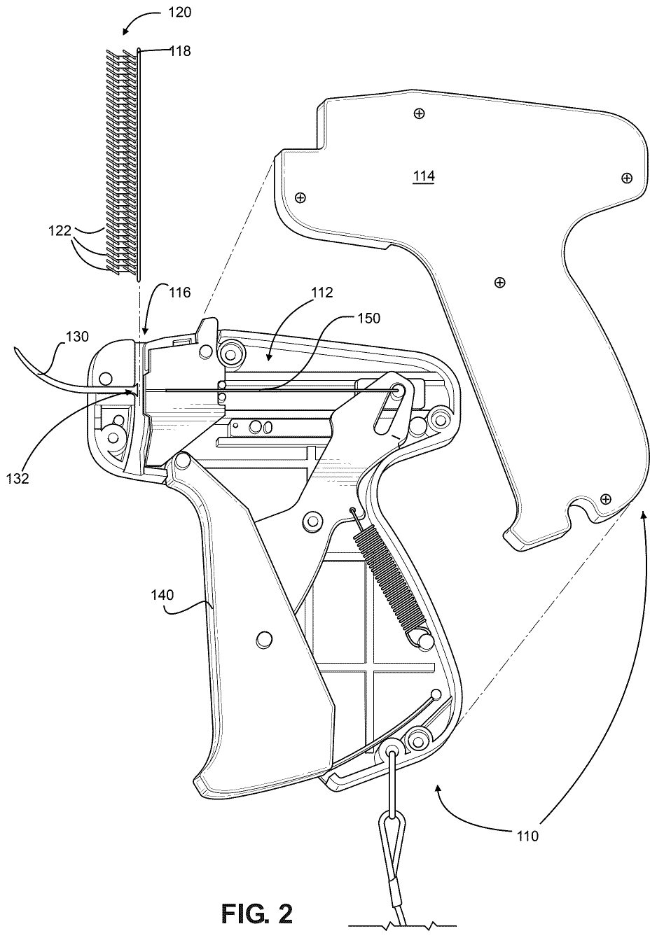

Turning to FIG. 2, the suture applicator shell 110 may define an inside 112 and an outside 114 configured to securely mount the hollow surgical needle 130. Means for driving individual stitches through the hollow surgical needle are provided. For instance, in some embodiments, a mechanical actuator 140 may be disposed on or otherwise accessible from the outside 114 of the shell and operatively connected to a stitch driver 150 housed within the inside 112 of the shell. Likewise, means for feeding the stitch clip 120 into the inside 112 of the shell may be provided. In some embodiments, such means comprises a retention mechanism, such as groove 116 disposed on a portion of the shell which is configured to guide the stitch clip 120 inside the applicator to align an individual stitch filament 122 with an open end 132 of the hollow surgical needle 130. Indeed, it may be seen that the individual stitch filaments may be anchored to and removable by the force of the stitch driver 150 from a spine 118.

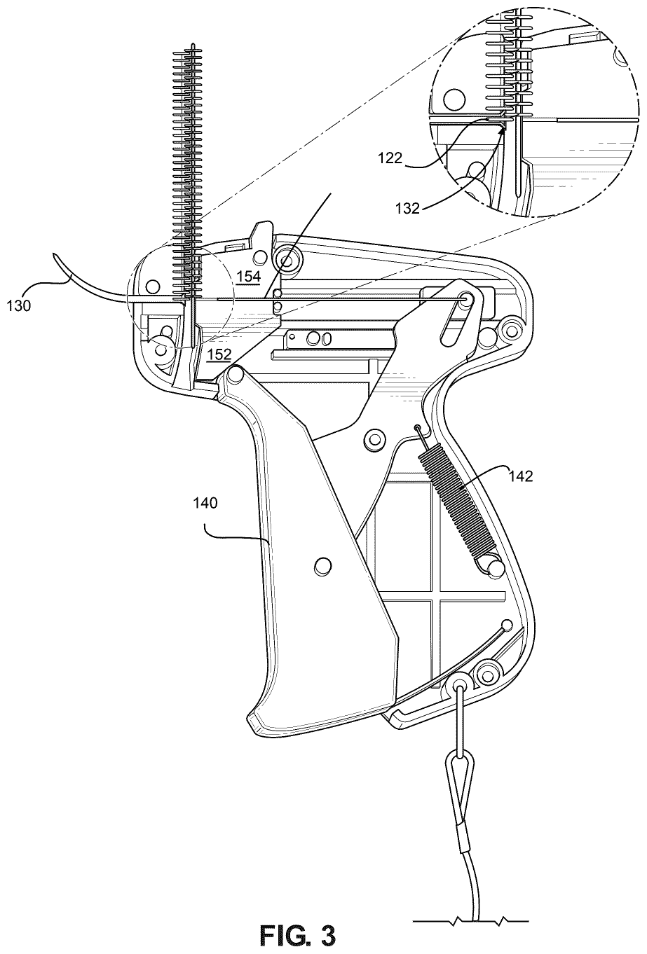

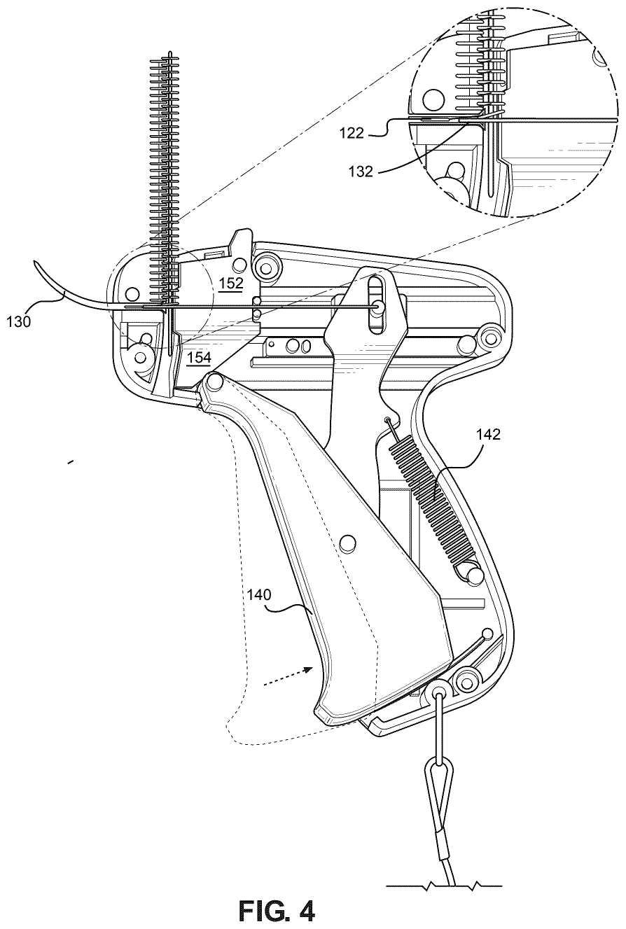

This alignment may be more clearly visible with reference to FIGS. 3 and 4. That is, in some embodiments, a mechanical actuator 140 is operative to advance the stitch driver 150 into an end 132 of the hollow surgical needle 130, such that the stitch driver 150 successively engages one or more of the elastic, biocompatible filaments 122 from the stitch clip and deploys the filament through the hollow surgical needle 130 into opposing sides of lacerated human or animal tissue. It is contemplated that means for guiding the stitch driver 150 into the end 132 of the hollow surgical needle 130 may be provided as well. One of ordinary skill in the art will recognize that such means may take innumerable forms. However, for the sake of non-limiting example only, it may be seen that a pair of opposing plates 152, 154 may be secured within the shell 110 of the suture applicator in order to precisely guide the movement of the stitch driver 150.

The stitch driver 150 may be any material sufficiently strong enough to successively press individual suture filaments 122 through the hollow needle. It is contemplated, however, that the stitch driver 150 be flexible and resilient enough to bend through any interior curve defined by the hollow surgical needle 130 and return to its original shape when retracted.

In some embodiments, the mechanical actuator 140 of the device may be integrated with a depressable button or trigger accessible from the outside of the applicator shell. It is contemplated that when the button or trigger is engaged, as in FIG. 4, it may directly activate any internal mechanism to advance the stitch driver, deploy an individual filament 122 from the clip through the hollow surgical needle 130, and reset the system for subsequent operations such as by action of a spring 142. This integration simplifies the operation of the device, enabling precise control over the deployment process while reducing the number of components required. The compact configuration enhances ergonomic handling, making the device intuitive and efficient for suturing applications in both human and animal tissue.

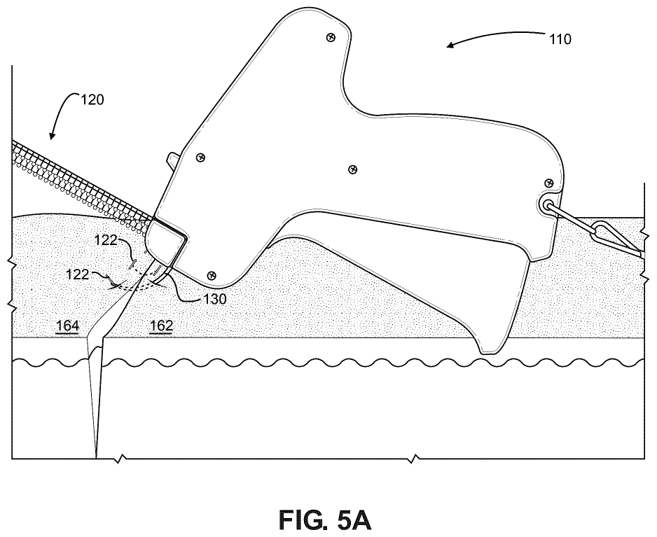

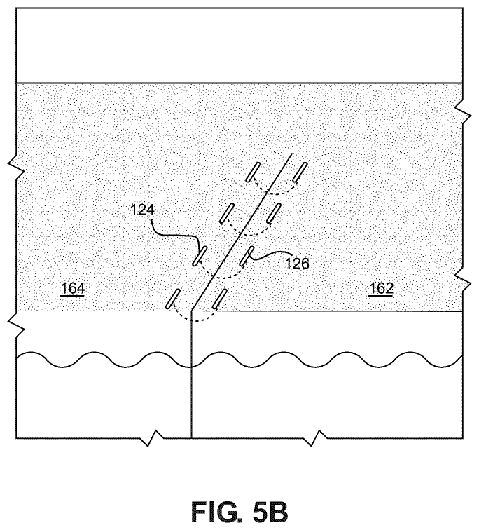

The hollow surgical needle 130 may be any shape or size as desired to needed to guide an individual suture filament from an outside of one laceration border 162, underneath the dermis, and then to an outside of an opposing laceration border 164, as shown, for example, in FIGS. 5A and 5B. In some embodiments, a front end of the hollow surgical needle (somewhat obscured from view, but known to those of ordinary skill in the art) may be beveled, or sharpened to an angular tip to reduce force needed to puncture tissue and further reduce the amount that the needle may deflect when inserted into tissue. The particular bevel length, bevel angle and inclination angle will not limit the invention. Notably, the hollow surgical needle 130 may define a curve amounting to some fraction of a circle. In some embodiments, the hollow surgical needle 130 may define a compound curve or even a J-shape. Thus, it will be understood that the particular degree of needle curvature will not limit the invention.

Once a user, such as veterinarian or physician or medic, guides the hollow surgical needle 130 from makes an initial needle stick from outside of one laceration border 162, underneath the dermis, and then to an outside of an opposing laceration border 164, such user may engage the means for driving individual suture filaments (not shown here) in order to push an individual suture filament 122 from the stitch clip 122 engaged with the stitch applicator 100 through one end of the hollow surgical needle 130 (within the shell) and out the opposing beveled end.

Turning to FIG. 5B in particular, each filament 122 comprising the individual stitches themselves comprise an elastic, biocompatible, and medically acceptable structure with a central connecting segment (obscured by the lacerated epidermis) and two opposing elongate legs 124, 126 extending from the central segment. When deployed, the first elongate leg 124 is driven through the hollow surgical needle and emerges on the exterior of one side of the lacerated tissue 164. The central segment is positioned beneath the tissue, preferable underneath the dermis, while the second elongate leg 126 emerges on the exterior of the opposing side 162 of the laceration. This configuration secures the tissue by anchoring the filament on both external sides of the laceration and maintaining the central segment beneath the tissue.

The filament 122 may comprise any suitable biocompatible material as needed or desired to repair lacerations in human or animal tissues. For example, absorbable monofilament sutures may be utilized for being characterized by high flexibility and elasticity. These features may be useful when suturing particularly fragile tissues. Braided or twisted multifilament sutures are also contemplated and may be selected for their higher memory and low bacterial adhesion. As above, the particular type, size, construction, and quality of the filament 122 selected to define individual stitches will not limit the invention and may vary depending on the needs and preferences of the user and the lacerated subject.

It is well known that the number of stitches required per inch for a wound can vary depending on several factors, including the type and severity of the wound, the location on the body, and the preference of the individual performing the suturing. For example, smaller and more delicate wounds may require more stitches per inch to ensure proper closure and healing. On the other hand, larger and deeper wounds may require fewer stitches per inch due to the need for wider sutures to hold the tissues together effectively. Thus, the number of stitches applied per inch may be determined by a healthcare professional based on their own clinical judgment and assessment of the wound based on factors such as the tension on the wound edges, the type of suture material used, and the desired cosmetic outcome. Because of this, it is contemplated that the type, size, and number of filaments provided along the stitch clip may vary without departing from the invention.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### CONCLUSIONS, RAMIFICATIONS, AND SCOPE

While certain embodiments of the invention have been illustrated and described, various modifications are contemplated and can be made without departing from the spirit and scope of the invention. For example, the relative positioning and orientation of the actuator, trigger, and the curvature of the surgical needle may be adjusted or configured differently to accommodate specific needs or preferences. Accordingly, it is intended that the invention not be limited, except as by the appended claim(s).

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the suture applicator with which that terminology is associated. In general, the terms used in the following claims should not be constructed to limit the suture applicator device and system to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the suture applicator device and system are not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the suture applicator device and system.

What is claimed is:

1. A device for suturing lacerations in human or animal tissue, comprising:
a shell defining an inside and an outside and configured to securely mount a hollow surgical needle;
a mechanical actuator disposed on the outside of the shell and operatively connected to a stitch driver housed within the interior of the shell; and
means for feeding a stitch clip comprising a plurality of elastic, biocompatible filaments as individual stitches removably disposed along a spine into the interior of the shell;
wherein the mechanical actuator is operative to advance the stitch driver into an end of the hollow surgical needle, such that the stitch driver engages one or more of the elastic, biocompatible filaments from the stitch clip and deploys the filament through the hollow surgical needle into opposing sides of lacerated human or animal tissue.

2. The device of claim 1, wherein the means for feeding the stitch clip is a retention mechanism configured to hold the stitch clip in position during operation.

3. The device of claim 1, further comprising an ergonomic grip comprising a trigger operatively connected to the actuator, enabling controlled, single-handed operation for precise placement of individual stitches.

4. The device of claim 1, further comprising means for guiding the stitch driver into an end of the hollow surgical needle.

5. A system for suturing lacerations in human or animal tissue, comprising:
a shell defining an inside and an outside;
a hollow surgical needle securely mounted to the shell;
a stitch clip comprising a plurality of elastic, biocompatible filaments as individual stitches removably disposed along a spine;
a mechanical actuator disposed on the outside of the shell and operatively connected to a stitch driver housed within the interior of the shell; and
means for feeding the stitch clip into the interior of the shell;
wherein the mechanical actuator is operative to advance the stitch driver into an end of the hollow surgical needle, such that the stitch driver engages one or more of the elastic, biocompatible filaments from the stitch clip and deploys the filament through the hollow surgical needle into opposing sides of lacerated human or animal tissue.

6. The system of claim 5, wherein the means for feeding the stitch clip is a retention mechanism configured to hold the stitch clip in position during operation.

7. The system of claim 5, further comprising an ergonomic grip comprising a trigger operatively connected to the actuator, enabling controlled, single-handed operation for precise placement of individual stitches.

8. The system of claim 6, further comprising means for guiding the stitch driver into an end of the hollow surgical needle.

9. A kit for suturing lacerations in animal or human tissue, comprising:
in sterile packaging, at least one stitch clip comprising a plurality of elastic, biocompatible filaments as individual stitches removably disposed along a spine and at least one hollow surgical needle; and
a suture applicator, comprising,
a shell defining an inside and an outside;
means for securely mounting the hollow surgical needle to the shell;
a mechanical actuator disposed on the outside of the shell and operatively connected to a stitch driver housed within the interior of the shell;
wherein the mechanical actuator is operative to advance the stitch driver into an end of the hollow surgical needle, such that the stitch driver engages one or more of the elastic, biocompatible filaments from the stitch clip and deploys the filament through the hollow surgical needle into opposing sides of lacerated human or animal tissue.

10. The kit of claim 9, wherein the means for feeding the stitch clip is a retention mechanism configured to hold the stitch clip in position during operation.

11. The kit of claim 9, further comprising an ergonomic grip comprising a trigger operatively connected to the actuator, enabling controlled, single-handed operation for precise placement of individual stitches.

12. The kit of claim 9, further comprising means for guiding the stitch driver into an end of the hollow surgical needle.
